(19) United States
(12) Patent Application Publication
(10) Pub. No.: US 2005/0010121 A1
(43) Pub. Date: Jan. 13, 2005

(54) System and method for detecting and analyzing electrocardiological signals of a laboratory animal

(76) Inventors: William Payne Ross, Saranac Lake, NY (US); Daniela Brunner, Riverdale, NY (US); David Arthur LaRose, Pittsburgh, PA (US)

Correspondence Address: MINTZ LEVIN COHN FERRIS GLOVSKY & POPEO, 666 THIRD AVENUE, NEW YORK, NY, 10017, US

(21) Appl. No.: 10/616,069

(22) Filed: Jul. 9, 2003

(51) Int. Cl. A61B 5/0402

(52) U.S. Cl. 600/509

(57) ABSTRACT

Methods and systems for detecting a signal indicative of at least a heart beat, a heart rate, or an ECG waveform of an animal is provided. The systems and methods may include scanning each of a plurality of electrodes for a signal indicative of contact by an animal and selecting a signal from each of at least a pair of electrodes, where each selected electrode includes a signal indicative of contact with the animal. The systems and methods may also include creating a differential signal from the signals of at least a pair of electrodes and determining at least a heart beat, a heart rate, or an ECG waveform from the differential signal.

### FIELD OF THE INVENTION

The present invention is related to a methods and systems for non-invasively detecting an ECG of a laboratory animal, and more particularly, to methods and systems for non-invasively detecting at least one of a heart beat, heart rate and one or more ECG waveforms (and/or parameters thereof) of a laboratory animal via a plurality of electrodes, contained preferably within an enclosure.

### Background of the Invention

Animals in general, and rodents in particular, have long been used in biomedical research of human disease conditions and therapeutics. In that regard, the mouse is probably the most extensively used animal in biomedical research. Mice are the animals of choice for experimentation because of their small size, short reproductive cycle, and the breadth of knowledge accumulated about mice and their biology.

As the human and mouse genome mapping projects have been more or less completed, non-invasive measurement of physiological parameters in mice is highly desirable. For example, measurement of heart rate, heart rate variability, and electrocardiogram (ECG) indices have, for nearly a century, provided clinicians with important diagnostic tools. Such data in mice may provide valuable information regarding the roles of genes and drugs in human disease. More specifically, in order to observe the effect of pharmaceutical drug classes on heart rate in mice and to obtain data for use as additional identifying metrics in a data-mining process, it is necessary to capture some form of ECG information.

When testing a drug on a mouse, for example, the drug generally has an effect on one or more biological, physiological and behavioral aspects of the mouse. Such effects on these aspects almost always occur simultaneously. Thus, not only is the ECG of the mouse monitored, but also the timing and nature of physical mouse movements. For example, one drug may have an effect of making the heart beat faster, but the animal may not move much; another drug may make the heart beat faster but, instead, also increase the activity of the mouse. Thus, to obtain a more comprehensive drug profile, it is advantageous to allow for an area in the experimental enclosure that permits the animal to move around.

Although some prior art methods and devices allow for the accurate detection of an animal's ECG in a large enclosure, they are highly invasive, in that electrodes are usually implanted into or glued onto the animal, with the signal wires coming from/out of the body of the animal to a connector device. Such devices disadvantageously interfere with mouse movements thereby disguising drug effects on behavior.

Vetterlein et al. (Am J Physiol 247:H1010-H1012; 1984) describe a method for measurement of heart rate in awake, non-instrumented rats. In their paper, they describe detection of the heart rate in a rat by placing the rat in a small enclosure within a plastic 4-sided cage with built-in metal plates. A manual switch was activated to record heart rate when it was determined that a front leg and a back leg were touching two pads. Such a system also disadvantageously restricts mouse movements.

U.S. Pat. No. 6,445,941 (Hampton et al.), herein incorporated by reference, discloses an automated method of detecting and recording a mouse ECG, with non-invasive electrodes. The system detects the heart rate of the animal when the animal touches at least three of four electrodes within a small enclosure. However, the disclosed system and method are extremely limited in design and application. First, Hampton et al. is limited in the number of electrodes that may be used to obtain the ECG. Any more than four electrodes and the circuitry becomes complex and unreliable. Moreover, radio frequency interference coupled with the very low ECG voltage present at the paws (for example) of the mouse (in the 100 micro-volt range), of Hampton et al., may compromise the reliability of the ECG data, or the ability to obtain any data at all.

Given that practical application of the Hampton et al. device limits the number of electrodes to four electrodes and the animal must touch at least three of the electrodes, it necessarily requires that the mouse be placed in a very small enclosure (relative to the size of the animal being tested), using only four electrodes so that the mouse always is in contact with at least three of the electrodes. This design also does not prevent long periods without a good ECG signal, in cases when the mouse reaches immobility in a wrong position (i.e. without touching the electrodes). Moreover, using such a small enclosure limits the ability to accurately gauge behavior of the mouse that may only be exhibited in a larger enclosure. Thus, such behavioral observations cannot be successfully accomplished together with such non-invasive ECG apparatuses.

Accordingly, there exists a present need for a device and method to non-invasively monitor and record ECGs in a laboratory animal in a large area enclosure so that multiple biological, physiological and behavioral aspects of the laboratory animal can be tracked simultaneously.

### SUMMARY OF THE INVENTION

The present invention presents novel systems and methods for accurately and non-invasively detecting an ECG of a laboratory animal, and one or more parameters thereof, using a number of electrodes in any size enclosure. The electrodes (or sensor pads as used in the present description) may be closely coupled with detection and/or processing circuitry to quickly boost signal levels of the electrodes. This may be advantageously accomplished, for example, by making the floor of an animal test enclosure a printed circuit board (PCB) with the electrodes being on the top of the board, and circuitry mounted on the bottom of the board. The electrodes may also be mounted on movable columns/towers, which force the laboratory animal to make contact with at least two or more electrodes at once. The mounting of the electrodes on columns also alleviates the electrodes coming into contact with any excretion made by the animal, and permits removal and cleaning of the electrodes without disturbing the animal.

Accordingly, in one embodiment of the present invention, an apparatus for detecting a signal indicative of at least one of a heart beat, a heart rate, and one or more ECG waveforms of an animal may include a first multiplexer for receiving a signal of each of a plurality of electrodes capable of being contacted by a part of an animal for a period of time. The first multiplexer includes a first output comprising the signal of a first electrode of the plurality of electrodes. The apparatus may also include a second multiplexer for receiving a signal of each of the plurality of electrodes, where the second multiplexer includes a second output comprising the signal of a second electrode of the plurality of electrodes. The apparatus may further include a differential circuit for receiving the first output of the first multiplexer and the second output of the second multiplexer. The differential circuit may include a differential signal based upon the first output of the first multiplexer and the second output of the second multiplexer. The differential signal may be indicative of at least one of a heart beat, heart rate and an ECG waveform of an animal.

In another embodiment of the present invention, a method for detecting a signal indicative of at least one of a heart beat, a heart rate, and one or more ECG waveforms of an animal may include scanning each of a plurality of electrodes for a signal indicative of contact by an animal and selecting a signal from each of at least a pair of electrodes. Each selected electrode includes a signal indicative of contact with the animal. The method may also include creating a differential signal from the signals of at least two electrodes and determining at least one of a heart beat, a heart rate and one or more ECG waveforms from one or more differential signals.

In another embodiment of the present invention, a system for detecting at least one of a heart beat, a heart rate and an ECG of an animal may include means for scanning each of a plurality of electrodes for a signal indicative of contact by an animal and selecting means for selecting a signal from each of at least a pair of electrodes. Each selected electrode may include a signal indicative of contact with the animal. The system may also include creating means for creating a differential signal from the signals of the at least a pair of electrodes and determining means for determining a heart beat, a heart rate and/or one or more ECG waveforms from one or more differential signals.

In yet another embodiment of the present invention, an apparatus for detecting at least one of a heart beat, a heart rate and one or more ECG waveforms of an animal may include a plurality of electrodes spaced apart from one another a predetermined distance and positioned on columns. Each electrode passes a signal indicative of a heart beat of the animal upon the presence of a part of the animal in contact with an electrode.

In still yet another embodiment of the present invention, a method for detecting a signal indicative of at least one of a heart beat, a heart rate, and one or more ECG waveforms of an animal may include scanning a plurality of electrodes, each of which may be in contact with a part of an animal, over a predetermined time period. Scanning may include computing the maximum of absolute values of substantially all the electrode signals during the predetermined time period, determining at least a first pair of electrodes signals having the highest maximum value relative to other electrode signals, determining whether the signals from the first pair of electrodes are greater than a predetermined threshold value and determining a differential value from the signals of the first pair of electrodes upon the value of the signals being greater than the threshold. The method may also include capturing a plurality of differential values via scanning, wherein the captured differential values represent a waveform, and processing the waveform.

In the above embodiment, processing may include determining a frequency distribution of the waveform, comparing the frequency distribution of the waveform to a predetermined frequency distribution of a predetermined ECG waveform, comparing the maximum and/or mean amplitude of the waveform to predetermined maximum and/or mean amplitude values of the predetermined ECG waveform upon the frequency distribution of the waveform coming within the frequency distribution of the predetermined ECG waveform and returning to the capturing step upon reaching the maximum amplitude value corresponding to the maximum amplitude value of the predetermined ECG waveform and/or the mean amplitude value of the waveform corresponding to the mean amplitude value of the predetermined ECG waveform.

Further yet, the above method embodiment may also include:
returning to the scanning step if the maximum amplitude value fails to correspond to the maximum amplitude value of the predetermined ECG waveform and/or the mean amplitude value of the waveform fails to correspond to the mean amplitude value of the predetermined ECG waveform; and/or
subsequently scanning of the electrodes upon the determination that the signals from the first pair of electrodes are less than a predetermined threshold value.

Moreover, computing the absolute values in this method embodiment may include acquiring a sample signal representing a voltage sample from a first electrode, calculating the absolute value of the sample signal of the first electrode and storing the absolute value of the sample signal for the first electrode as a new maximum upon the absolute value of the sample signal being the largest for the first electrode.

The invention may also include computer readable media embodiments for performing one or more of the methods of the present invention. The invention may also further include computer application program embodiments for enabling a computer system to perform one or more of the methods.

These aspects and advantages of the invention will become even clearer with reference to the drawings, a brief description of which is set out below, and the detailed description which follows.

### BRIEF DESCRIPTION OF THE DRAWINGS

FIG. 1 is a block diagram illustrating an overview of a system for detecting an ECG of a laboratory animal according to an embodiment of the present invention.

FIG. 2 is a schematic block diagram of a circuit for detecting the ECG of an animal according to an embodiment of the present invention.

FIG. 3 is a flowchart illustrating a process of detecting a signal indicative of an ECG waveform using, for example, the circuit shown in FIG. 2.

FIGS. 4-14 illustrate circuit diagrams for various components of a system according to some of the embodiments of the present invention.

FIG. 4 illustrates one example of a power supply circuit.

FIG. 5 illustrates an example of a pad circuit for picking up an electrical signal of a laboratory animal.

FIG. 6 illustrates one example of muliplexer devices for detecting ECG signals.

FIG. 7 illustrates one example of a second-stage differential amplifier.

FIG. 8 illustrates one example of an electrical filter.

FIG. 9 illustrates one example of a third stage amplifier.

FIG. 10 illustrates one example of an analog-to-digital converter, multiplexer and control circuit.

FIG. 11 illustrates one example of a processor.

FIG. 12 illustrates one example of processor parallel ports.

FIG. 13 illustrates one example of an LED array circuit for displaying ECG waveforms, diagnostics, and the like.

FIG. 14 illustrates one example of a connector for connecting the circuit to a computer system.

FIG. 15 is a chart illustrating the superimposed waveforms of ECGs obtained simultaneously from a laboratory animal via an embodiment of the present invention and a standard subcutaneous electrode system.

FIG. 16 is a three-dimensional chart illustrating the results of a comparison test of detecting heart rate of a laboratory animal simultaneously using a method and system according to an embodiment of the present invention and standard subcutaneous implanted electrodes with regard to a baseline heart rate, and heart rates upon administering two different drugs to the animal.

### DETAILED DESCRIPTION OF THE EMBODIMENTS

It is understood that some embodiments of the present invention (or elements thereof) may be carried out using computing systems and devices (servers, personal computers, mainframes, minicomputers, super computers and the like, networked and stand-alones), as well as their associated peripheral devices, and other devices with which such computer devices communicate. To that end, such computing devices generally include one or more processors for operating software (operating or otherwise), which thus may be used for carrying out one or more methods of the present invention. Moreover, such computer devices include RAM and ROM memory, hard drives, CD burners, flash memory, printers, input devices (e.g. keyboard, mouse, trackpad, microphone), sound devices (e.g. sound card, loudspeakers), networking devices (e.g., Ethernet) and the like.

Embodiments of the present invention may be used alone or in combination with a variety of laboratory devices for performing a variety of experiments. In that regard, the present invention may be used in combination with the laboratory systems disclosed in Published PCT application no. WO02/093318 and WO03/013429, the disclosures of which are herein fully incorporated by reference.

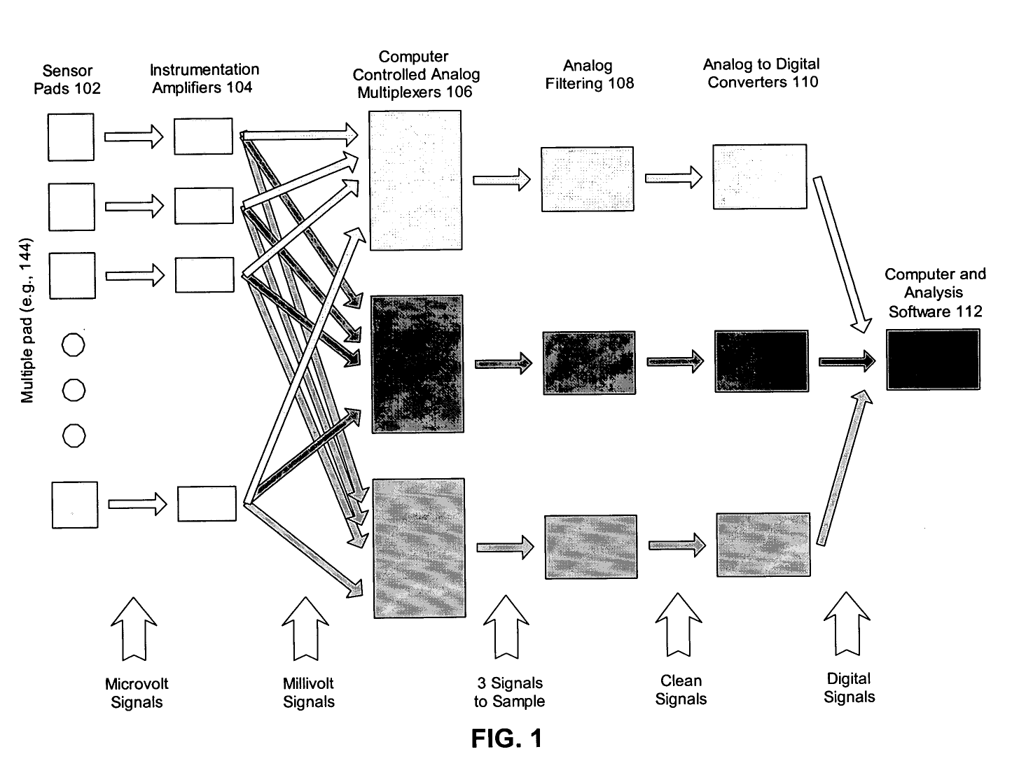

FIG. 1 illustrates a block diagram illustrating an overview of a system for detecting an ECG (and associated parameters thereof), according to some embodiments of the present invention. A plurality of sensor pads 102 (n number of pads) includes electrodes (not shown) that pick up an electrical signal indicative of the heart beat of a laboratory animal (e.g., a mouse). Reference to “mouse” in the present disclosure is used for exemplary purposes only and one of skill in the art will understand that the principles and embodiments of the present invention may be used to obtain ECG signals and data for any animal (including humans, for example).

Each sensor pad (n number) may be coupled to a respective instrumentation amplifier 104, (n number of amplifiers) to boost the electrical signals picked up by the respective sensor pad from, for example, a paw of the mouse. Such electrical signals from the pads are generally on the order of microvolts, which the amplifier boosts to the millivolt range (for example).

The output of each instrument amplifier may be connected to at least two or more multiplexers 106 (depending upon the number of pads present in the system). The multiplexers are used in combination with computer control, to scan each sensor pad to determine whether a signal emanating from a scanned pad is a heart beat signal from the mouse. A signal output from each multiplexer may be filtered (108), to eliminate unwanted electrical interference, from, for example, lights, motors (fans), and the like. Such a filter may therefore advantageously include, for example, a 50 Hertz and/or 60 Hertz low pass filter to remove electrical noise from AC devices.

The filtered signal(s) may then be sent to an analog-to-digital converter 110, where the analog signal is converted to a digital signal that may be forwarded to a computer system 112 for analysis.

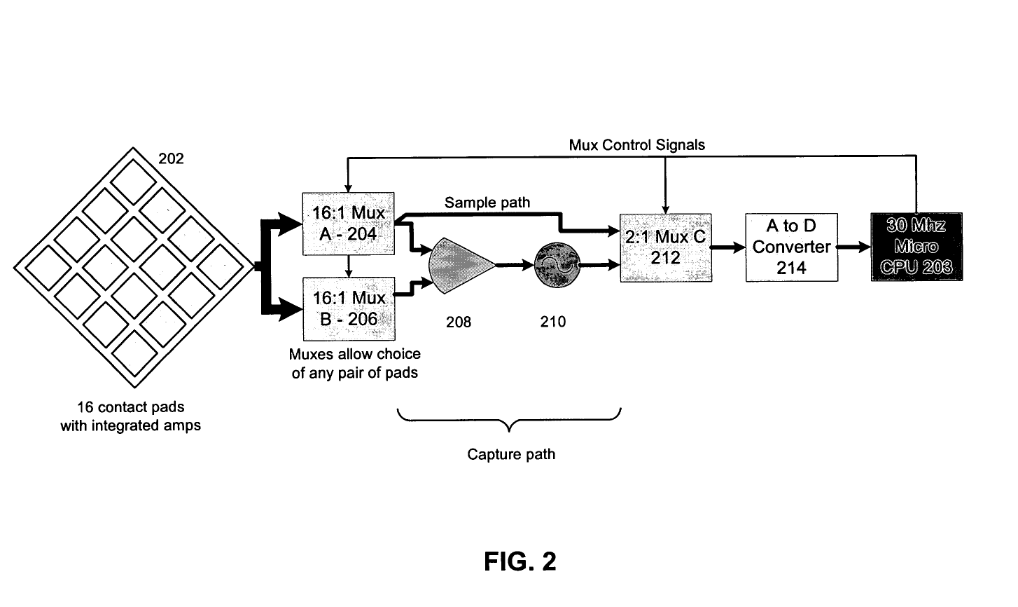

FIG. 2 illustrates an exemplary schematic diagram of an electronic circuit that may be used to detect ECG data signals of the mouse, using a 16-sensor pad system (for example). Of course, one of ordinary skill in the art will understand that this 16-pad system is merely representative, and that systems having more than 16 pads are easily implemented using the systems and methods according to the present invention. The 16-pad example may be used, for example, as a building block for a system representing multiples of 16 pads (e.g., 32, 64, 128, etc.). Of course, the underlying “building block” circuit may also be designed according to the present embodiment for 3 or more sensor pads (for example).

Accordingly, 16 contact pads 202 each having a corresponding electrode may be provided, where each electrode may be connected to a first stage amplifier (not shown). Each amplifier is preferably positioned immediately adjacent a corresponding electrode, so that the electrode may be immediately connected to the amplifier. This is done to limit the amount of exposed electrical conductor (e.g., wire), to minimize electrical interference picked up between the electrode and the amplifier. To further minimize any electrical interference therebetween, any exposed wire may be shielded.

A processor 203 may be used to process and analyze signals from each of the electrodes. Accordingly, the processor scans and selects signals from the sensor pads. To that end, signals from each of the first stage amplifiers are directed into multiplexer A (204) and multiplexer B (206), each of which may be a 16:1 multiplexer (for example). The processor controls the scanning of the sensor pad electrodes by the multiplexers and makes a determination as to whether a signal coming from a particular pad represents one that is representative of a part of the mouse (paw) touching the pad. Such detected signals may be signals with increased “noise.”

The output of each multiplexer may be directed into a differential “second stage” amplifier 208, which determines a difference in potential between the signals emanating from the multiplexers A and B, and amplifies it. The output of the differential amplifier may be filtered using a filter 210. The output of multiplexer A may also be directed to one input of a third multiplexer (multiplexer C) 212, which may be a 2:1 (or 16:1, or other) multiplexer, for example, which is also controlled by processor 203. An output of multiplexer C is directed to an analog-to-digital (A/D) converter 214, an output of which is ideally connected to the processor. This arrangement provides a feedback type mechanism for selecting one or more (preferably at least a pair) of electrodes, each of which having a signal indicating that the mouse has touched the selected electrode.

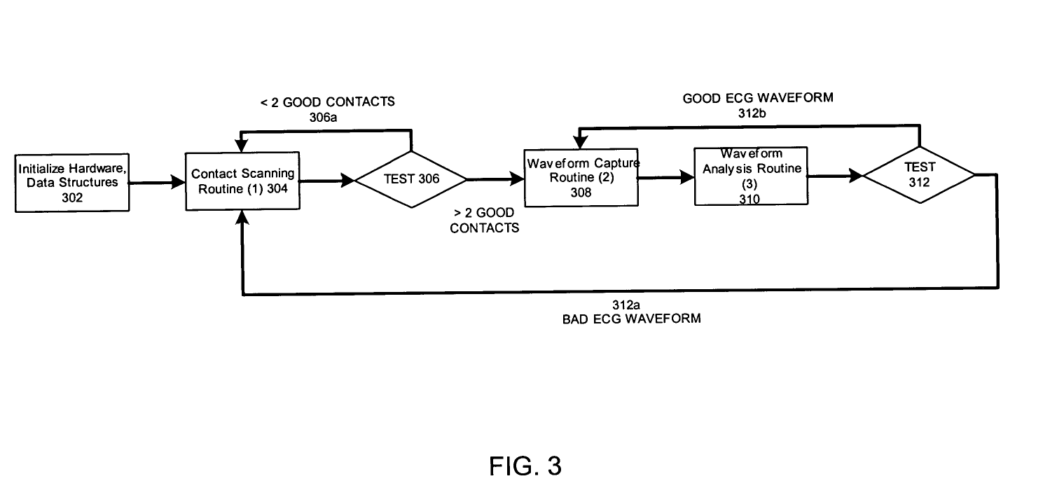

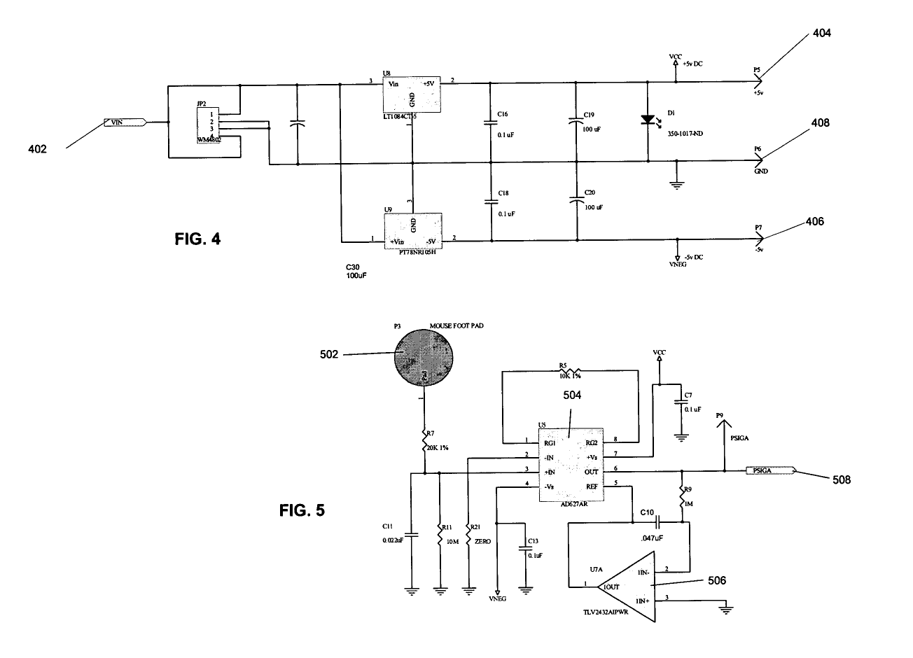

FIG. 3 represents an example of a process flow, operated on the processor, for capturing ECG data using the circuit of FIG. 2 (and/or FIGS. 4-14). Accordingly, the hardware and data structures are initialized (302). Scanning of the pads/electrodes is begun to seek a pad/electrode that has been contacted by a paw of a mouse (304). In that regard, the Contact Scanning Routine (outlined below) is started, which tests (306) each pad for contact by the mouse. This scanning is done until at least two pads being in contact with the mouse are determined. Accordingly, if less than two pads are determined to be in contact with the mouse, the scanning routine is run again (306a).

Upon the determination that at least two pads are in contact with the mouse (306b), a Waveform Capture Routine is started (308). The results of this routine (i.e., a captured waveform) are passed to a Waveform Analysis Routine (310). The Waveform Analysis Routine performs a test 312 where the captured waveforn is compared with a predetermined, expected ECG frequency distribution. For example, if the frequency distribution is a poor match, the process is returned to the Contact Scanning Routine 312b. Otherwise, a determination is made as to whether maximum amplitude and/or mean amplitude of the waveform are within a maximum amplitude and/or mean amplitude of a predetermined, expected ECG waveform of the particular laboratory animal. If not within the expected predetermined values, the process returns to the Waveform Capture Routine (312b).

Below are examples of an underlying process for each of a Contact Scanning Routine, a Waveform Capture Routine and a Waveforn Analysis Routine, referred to above, according to some embodiments of the present invention.

### Contact Scanning Routine

Setup multiplexer C to route output of multiplexer A directly to the A/D converter.

Compute maximum of absolute value of all pad amplifier outputs during a certain time period as follows:

While still more time available for scanning:
   For each contact pad:
      Set multiplexer A to take input from the pad amplifier
      Acquire a voltage sample from the pad via the A/D converter
      If the absolute value of the sample is the largest yet for this pad, save it as the new maximum
   End For Loop
End While Loop
   Determine the two pads with the highest maximums
   If both of the highest pads are above a user specified voltage threshold, Then
Drop out of the routine and pass maximum two pads on to Waveform Capture Routine
   Else
Return to top and scan again for maximum voltage values

### Waveform Capture Routine

Setup multiplexer A to route output of first maximum pad amplifier found in Contact Scanning Routine to the Differential Amplifier

Setup multiplexer B to route output of second maximum pad amplifier found in Contact Scanning Routine to the Differential Amplifier

Setup multiplexer C to route output of Noise Filter to the A/D converter

Acquire a number of amplified differential voltage samples from the A/D converter

Transmit voltage signals (ECG Waveform) to host computer over RS-232 serial port

Display ECG waveform on connected LCD display

Pass voltage samples on to Waveform Analysis Routine

### Waveform Analysis Routine

Compute fast Fourier transform on waveform to determine frequency distribution of signal

Compare peak frequency with expected ECG frequency distribution

If frequency distribution is a poor match

Return to Contact Scanning Routine
   Compare maximum and mean amplitude of waveform to expected values
   If amplitude is not within ECG norms
Return to Contact Scanning Routine
   Else
Return to Waveform Capture Routine to capture the next waveform

FIGS. 4-14 are circuit diagrams illustrating one example of a system for detecting an ECG of a mouse, using sixteen (16) electrodes. Such a system represents both a multistage system and process. One of ordinary skill in the art will understand that this circuit and the elements thereof represent only one such circuit for detecting an ECG using the methods described above and that other circuits may be designed which may include one or more different elements of the circuit(s) disclosed herein, or altered configurations including ordering of components, to perform a similar method. Moreover, one of skill in the art will also understand that the entire circuit, or components thereof, may be integrated into one or more microchips, for example. Further, the methods described above, especially those directed to the Contact Scanning, Waveform Capture and Waveform Analysis routines may be comprised in a hardwired circuit(s) or micro-chip(s). Most components of the electrical circuits detailed in FIGS. 4-14 may be obtained from most electrical component manufacturers including, for example, Texas Instruments, Inc., of Dallas, Tex., USA.

Accordingly, FIG. 4 illustrates an example of a power supply for the circuit. Input voltage may be between +7 and +20 volts (at several or more amps)(402) allowing the power supply to produce regulated power, at 5 volts at 5 amps (404), and −5 volts at 1 amp (406), for example, along with ground (408).

FIG. 5 represents an example of a sensor pad circuit 501 having an electrode footpad 502 made of, for example, an Ag/AgCl alloy. This circuit may be replicated for each electrode for the ECG data collection device. Each circuit may include a corresponding instrumentation amplifier 504 (e.g., AD627AR, Analog Devices of Norwood, Mass.) with a 25 times gain (for example), using a 10 kΩ resistor. Two sensor pad circuits may include a shared dual OP-AMP 506, for example (connected to REF pin 5 on AD627AR). This sets the instrumentation amplifier's reference voltage to approximately ground.

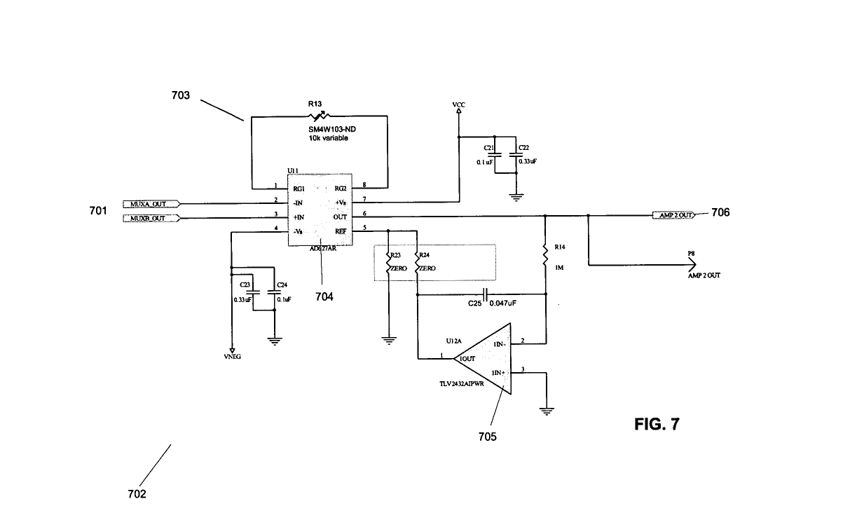

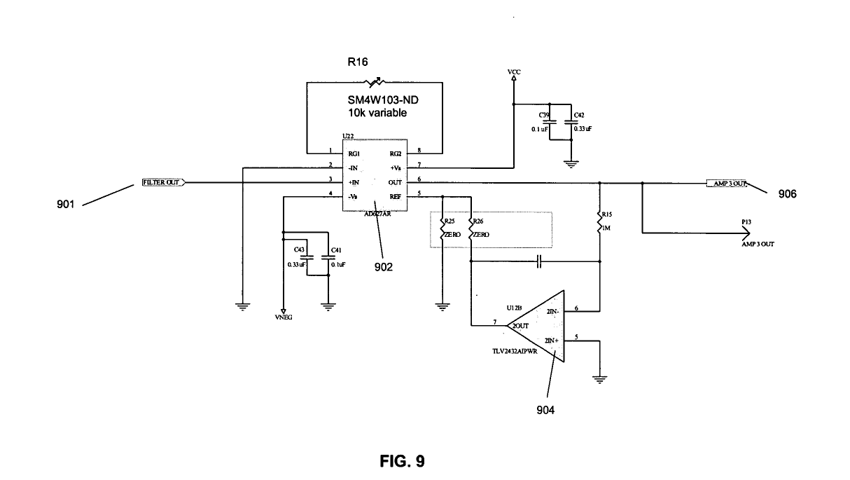

The adaptation speed of the OP-AMP in FIG. 5 is set to a slow rate by, for example, a 0.047 μF capacitor. The instrumentation amplifier also includes to inputs: −IN and +IN, for differential amplification, and may multiply the difference between the two signals by the set gain (e.g., 25×). For single-ended amplification, either −IN or +IN can be tied to ground. The present instrumentation amplifier may also be used in an additional element to the system for second and third stage amplification of a signal (see FIG. 7 and FIG. 9). Multiples of the present sensor pad circuit may be used to produce 16 sensor pads for one embodiment of the present invention, or any number of sensor pads. Accordingly, output from the circuit, 508, represents a signal from the sensor pad.

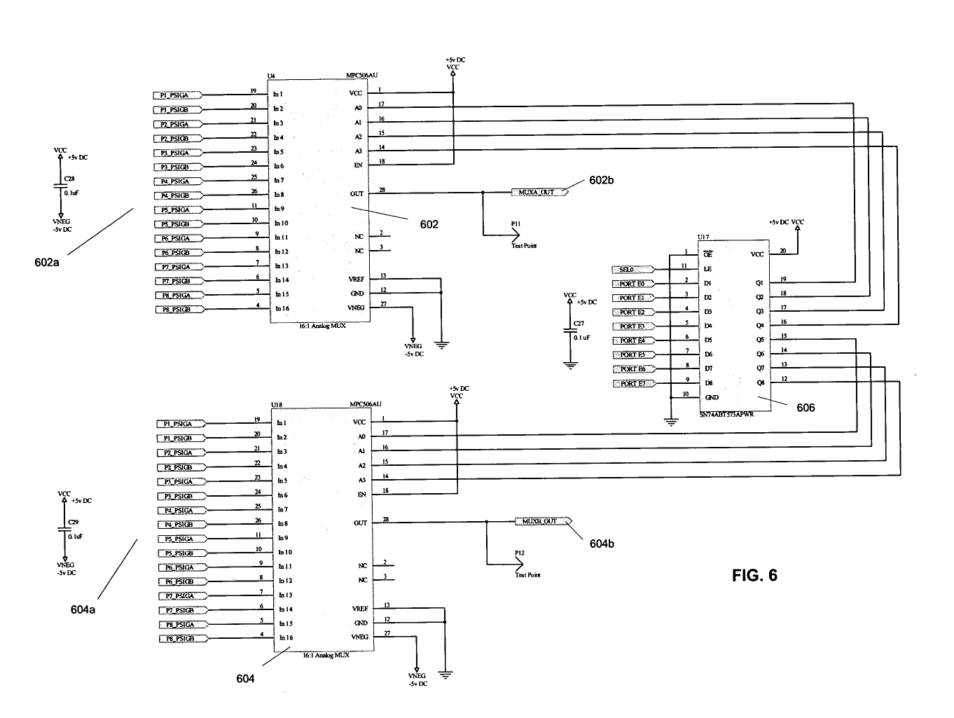

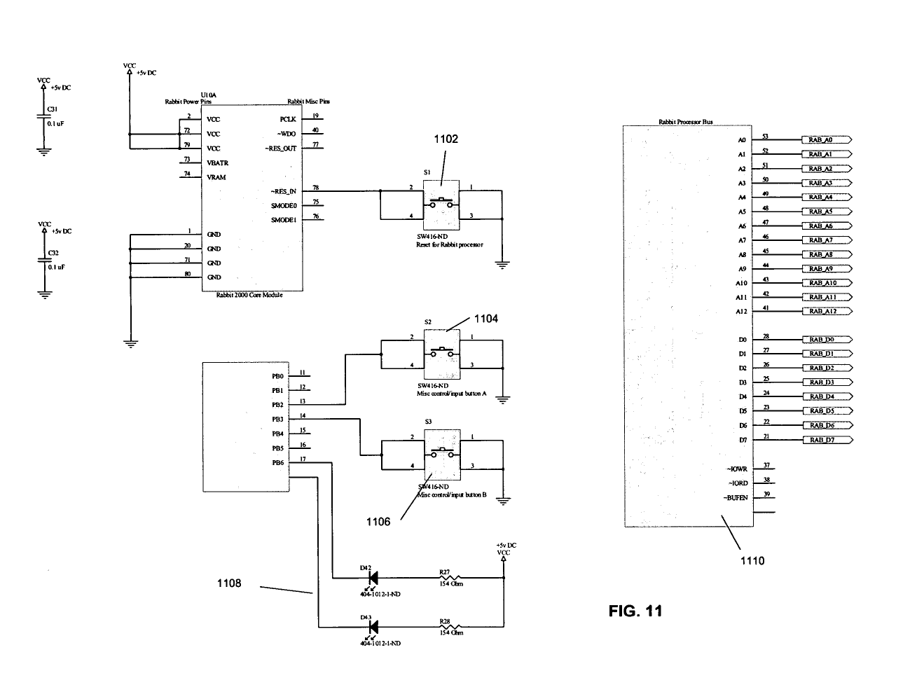

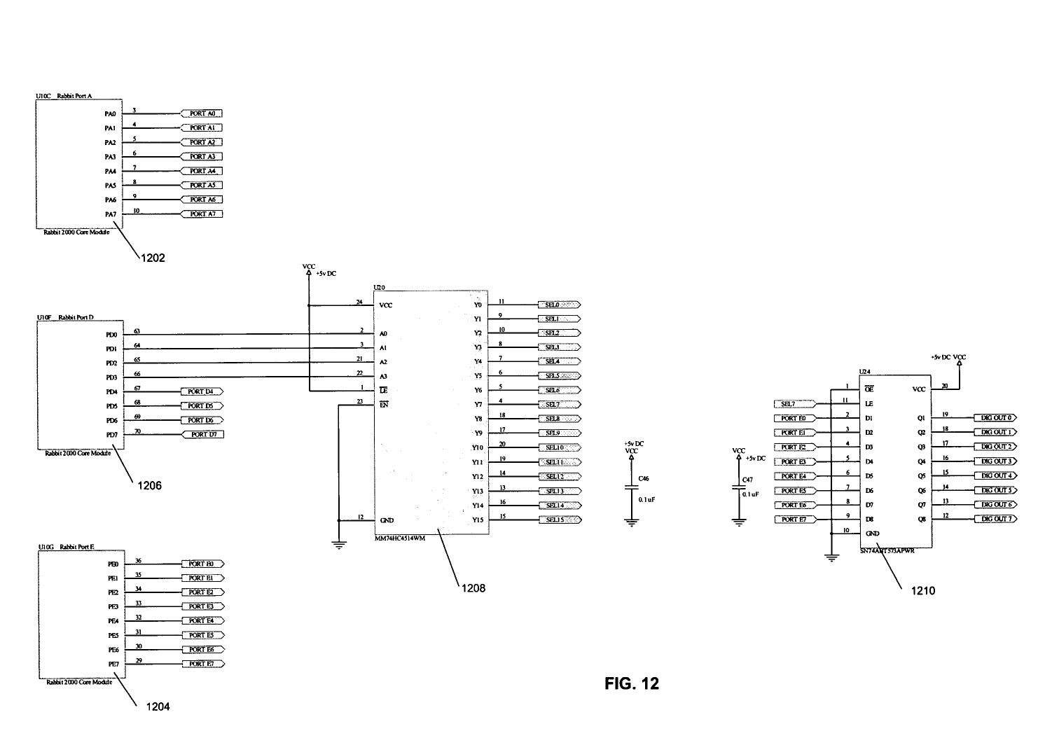

Each output of a sensor pad circuit is sent to two 16 input analog multiplexers (FIG. 6)(e.g., part No. MAX306CWI, from Maxim Communications Pte Ltd., of Singapore). As shown, inputs from each sensor pad are input to multiplexer A (602) at a respective input 602a, and are input to multiplexer B (604) at a respective input 604a. Each multiplexer includes an output: output A (602b) (multiplexer A), and output B (604b) (multiplexer B). A processor (FIGS. 11-12) controls both multiplexers with the aid of latch 606. The latch holds the address bits for the two multiplexers, and its input lines are tied to Port E on the processor (see FIG. 12).

Each multiplexer may sample, for example, the corresponding inputs 100 times each over a predetermined time period (for example) by each associated A/D converter, with the resulting 1600 signal choices being compared by the processor (FIG. 12). The 1600 sample signals may be unfiltered and have only been amplified by the first-stage amplifier on the electrode pad. The processor compares relative strengths of “noise” on the pads with respect to ground.

The outputs 701 of the multiplexers are received by a second stage, differential amplifier 702 (FIG. 7), which includes instrumentation amplifier 704 (AD627AR, for example) and OP-AMP 705. The OP-AMP may set the reference voltage for the second stage amplifier—which keeps the output 706 of the amplifier circuit output about ground. A cap value sets speed of adaptation, which is preferably set to a slow setting. A variable resistor 703 may be used to change the amplification of the signal (e.g., 205 Ω−1000× gain, 2.1 kΩ−100× gain, and 10 kΩ−25× gain). The now twice amplified signal (now referenced to Ground) is output via output 706.

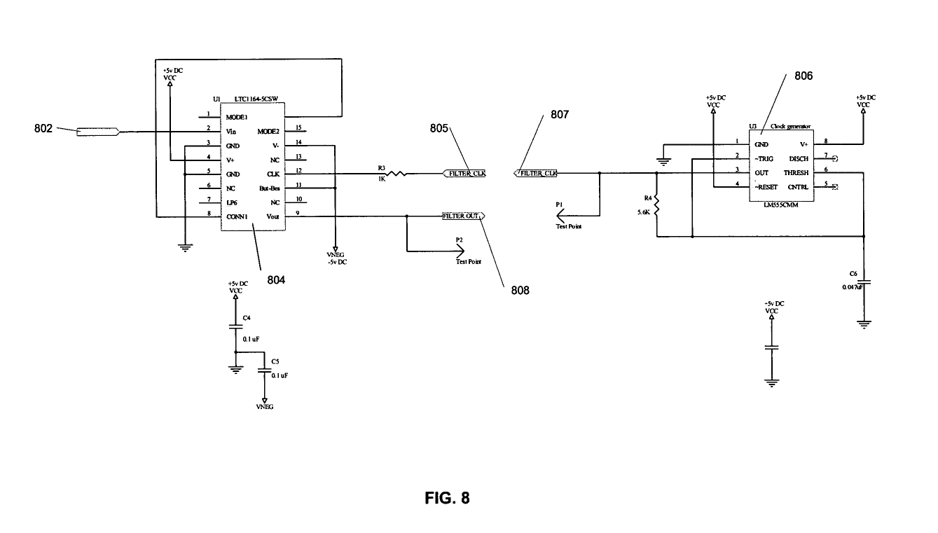

FIG. 8 represents components of a 60 Hz, low pass filter circuit for filtering out electrical interference from, for example, alternating current devices (e.g., lights, appliances, etc.). Accordingly, the output of the differential amplifier is input to the circuit at input 802. The circuit may include filter 804 (which may be an 8^{th} order Butterworth filter, with a cut-off frequency ratio of 1:100) and filter clock 806 which are connected via line 805 (filter) and line 807 (filter clock). The now filtered signal is output via output 808.

FIG. 9 represents a third-stage amplifier (optional), which may be similar to the first and second stage amplifiers and which may be positioned in the system circuit to receive the output of the filter circuit. Accordingly, the third stage amplifier may include an instrumentation amplifier 902, which receives the output of the filter via input 901. The third stage amplifier may also include an OP-AMP 904, which sets reference voltage at such a level to keep the amplifier output at about (for example) ground. An amplified signal (now three times amplified) is output via output 906.

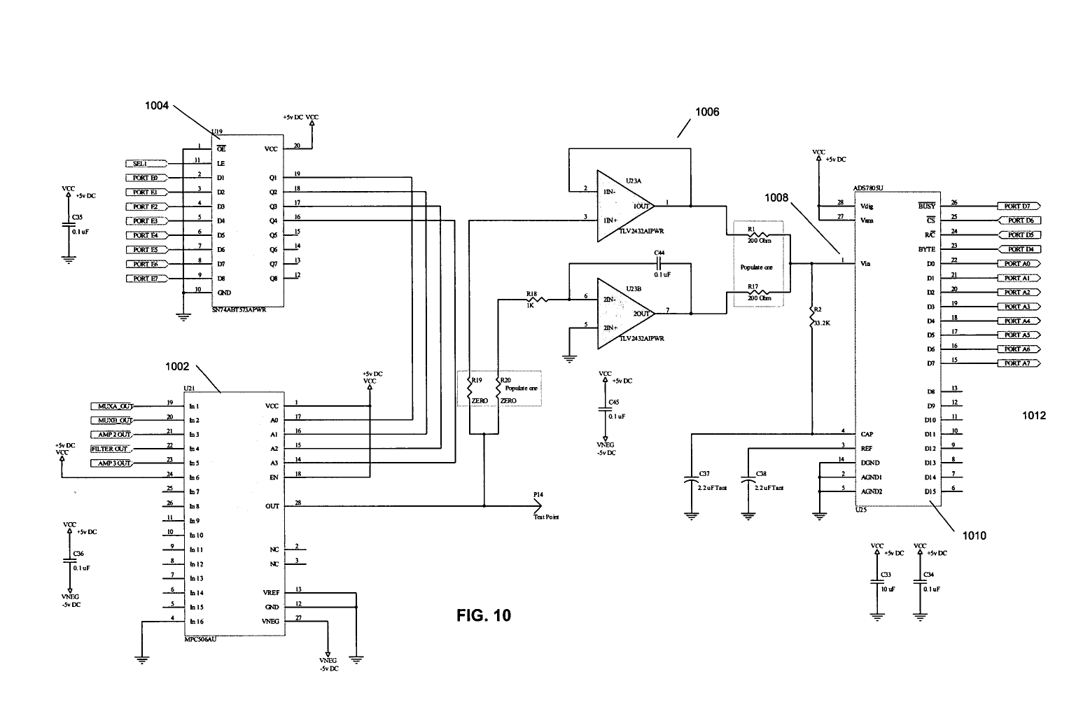

An A/D converter 1010 converts an analog signal from the filter (or third stage amplifier) to a digital signal. As shown in FIG. 10, a third 16-input multiplexer 1002 arbitrates between assorted output signals from multiplexing, filtering, and amplifying (e.g., the output from the differential amplifier—filtered/unfiltered, the output from at least one of the multiplexers) based on address data latched through latch 1004 from the processor. The third multiplexer passes a signal to the A/D converter (e.g., ADS7805U, Analog Devices of Norwood, Mass.), which takes the amplified, filtered analog signal and converts it to a 16-bit digital signal, the lower byte of which may then be passed to Port A of the processor. The control lines of the A/D converter, receive input from Port D of the processor (see FIG. 12).

Options for filtering and/or rectifying the signal, 1006, may be included prior to the signal being received by the A/D converter. The signal passing from the filtering and/or rectifying components then pass to the input 1008 of the A/D converter 1010. Outputs 1012 pass a digital signal to the processor.

FIG. 11 illustrates components of a processor that may be used with embodiments of the present invention. One such processor which may be used in the present circuit is a Rabbit 2000® Microprocessor (Rabbit processor), from Rabbit Semiconductor, of Davis, Calif., USA. In that regard, all the parameters, features, capabilities, functions, pin assignments and the like may be found in the Rabbit 2000® Microprocessor User's Manual 019-0069 (030307-H), which is herein incorporated by reference.

Several push buttons (1102, 1104 and 1106) may be included which allow for the reset of the processor, as well as other miscellaneous functions (e.g., testing, debugging, setting modes and the like) via test buttons 1104 and 1106. Similarly, LEDs 1108 may be used for testing, debugging, setting modes, etc. A system bus 1110 may be included to connect various elements of the present circuit to the Rabbit processor.

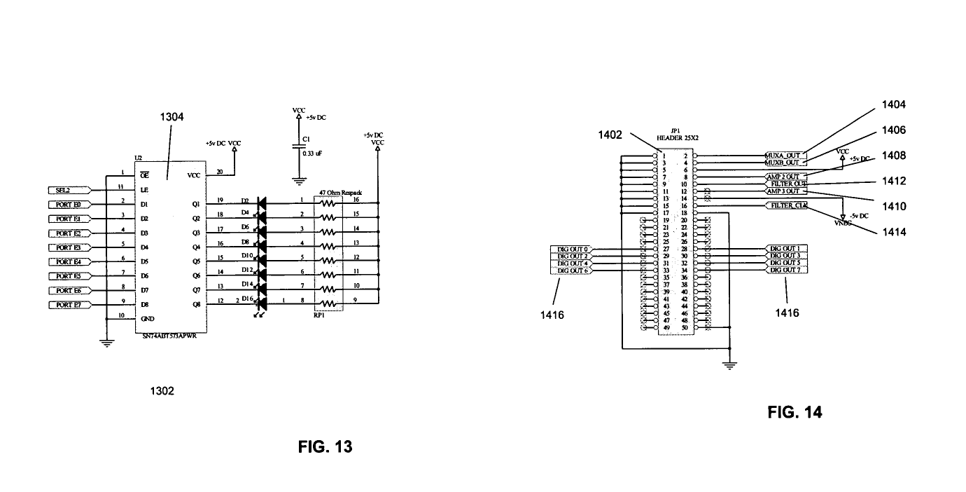

FIG. 12 illustrates parallel ports for the processor. As shown, port A (1202) may be dedicated to receiving input from the A/D converter. Port E (1204) may be used to output data for multiplexer control and LED (see FIG. 13) latches. Port D (1206) may be used in association with line decoder 1208, and for the source of the A/D converter's control lines. Preferably, the line decoder LOW is tied LOW, so that the decoder is always working, and latching is tied HIGH so that the address is never latched. The line decoder preferably generates the latch enable signals for the multiplexers and LED arrays (see FIG. 13).

The output of the processor may be sent to a computer for further processing and/or analysis via digital output port 1210. Such a computer may be connected to the present embodiment through any of parallel and/or serial connections, or any other communication means (e.g., infra-red, radio or other wireless, USB, Ethernet and the like). Moreover, such a computer may be used to control the present embodiment (for example).

One or more LED arrays may be used for diagnostics (e.g., show active pads, display scrolling text), or for displaying ECG parameters and details (e.g., heart beat, heart rate, ECG waveforms and waveform parameters). As shown in FIG. 13, each LED array 1302 may include 5 columns and 8 rows of LEDs, and each may include a latch 1340 for controlling each respective column of 8 LEDs. The latch bits are preferably set to zero (0) to turn the LEDs on, since each latch's LOW provides more current. Data inputs may be obtained from Port E of the processor, with the latch enabling signals being generated (for example) by the address line decoder (discussed above).

The exemplary circuit according to FIGS. 4-13 may also include a test connector as shown in FIG. 14. Accordingly, Header 1402 includes a plurality of ports (e.g., 50 ports), which may include connections to each multiplexer output 1404, 1406, each stage amplifier output 1408, 1410, the filter output 1412, the filter clock 1414, and various digital outputs 1416. The test connector may be made available for debugging and general input/output purposes. Among the various pins are one or more pins representing an 8-bit digital output from a third latch, which may serve as a buffer for passing data from Port E to an external PC or laptop (for example).

### ECG PERFORMANCE EXAMPLE:

A 14-day-old rat, with roughly the same weight and heart rate of an adult mouse, was used. The animal was anesthetized with ketamine and xylazene. Two stainless steel wound clips were attached to opposite sides of the animal's chest, approximately at the level of the heart. Wire leads from these chest leads were attached to the input cable of a Grass polygraph. The signal from these leads was amplified and filtered by a Grass EKG amplifier. The animals front paws were moistened with water and placed on the silver solder footpads on the devices circuit board. The front paws were resting on the pads without any additional pressure or even the full weight of the animal.

Outputs from the Grass driver amplifier and the foot ECG were put into a National Instruments A/D interface and data was acquired at 1000 samples/sec by special purpose software installed on a lap top computer. No attempt was made to equate the magnitude of the two amplified signals but they were fairly similar.

Approximately 8 minutes of data were acquired.

The digitized data were visually examined using a special purpose program that allows viewing of multiple signals and automated peak detection and marking. The R-waves in each waveform (i.e. foot vs. chest) were marked using this software. The signal quality was very good for both signals and few artifacts (i.e. missed beats or extra marks) were noted. After marking, two files were created; one with RR-intervals and one with the times at which each R-wave was marked.

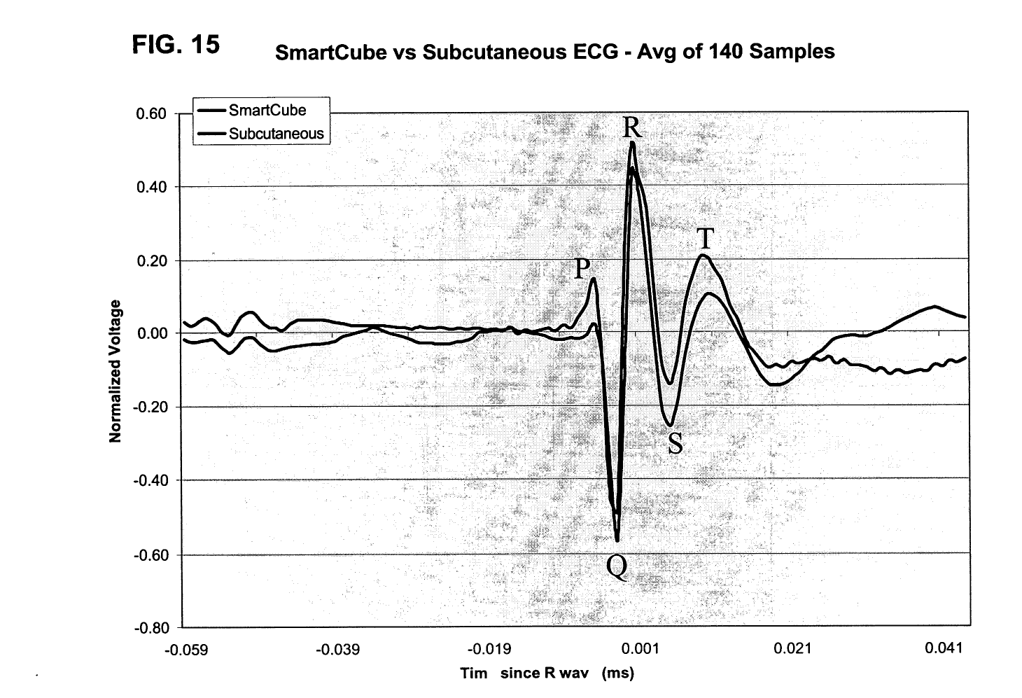

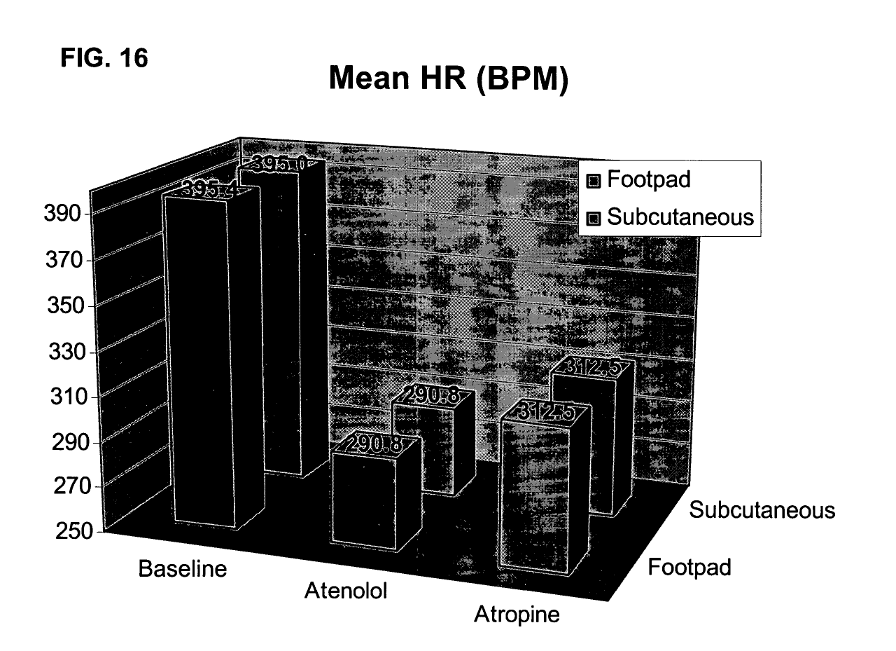

The first analysis used signal-averaging software to create composite waveforms from each signal. A 20 second sample containing 138 beats was used. Waveforms were averaged around the R-wave for 0.1 seconds before and after each R-wave. FIG. 15 shows the resulting composite waveforms from each source. As can been seen, the shapes of the ECG signals were virtually identical from the two sources. The form of the ECG was somewhat unusual due to the unconventional axis of recording. Although no P-wave could be discerned, the other components of the ECG were easily identified.

The RR-intervals were analyzed by a special purpose program that computes, in 30-second epochs, numerous parameters relating to central trend (means, and median) and RR-interval variability. The measures of variability are in both the time and frequency domain. For about the past 20 years there has been great interest in measures of heart rate variability as indirect indices of autonomic control (See References 1,2). These measures have proved to be very useful in developmental studies in both animals and humans (See References 3-5), adult psychophysiological studies (See References 6-8), and clinical cardiology (See References 9-10). Variation in heart rate (or RR-intervals) is created by fluctuations both sympathetic and parasympathetic activity to the heart. Of particular interest are measures of rapid, beat-to-beat variability as these are attributed to vagal (parasympathetic activity). In the following results we have computed the mean heart rate over several epochs, the standard deviation of RR-intervals within each epoch, and a time domain estimated of high frequency (i.e. ˜vagal modulation) variation. This latter measure, MSSD, is the root mean square of successive differences in RR-intervals.

FIG. 16 represents a 3D graph illustrating heart rates obtained simultaneously using an embodiment of the present invention and subcutaneous electrodes. Three heart rates were obtained: one baseline, and one each for the drugs atenolol and atropine. As the chart clearly indicates, the heart rates obtained using the present invention were virtually identical to those obtained using the subcutaneous electrodes.

Moreover, FIG. 16 indicates that ECG waveform parameters, and the associated intervals therebetween, were substantially identical for an ECG waveform obtained simultaneously using the present invention and subcutaneous electrodes.

Accordingly, the present invention presents improved systems and methods for non-invasively obtaining various data associated with an ECG of a laboratory animal. However, the present invention may advantageously be used with other systems and methods of analyzing an animal's biological, physiological and behavior aspects as well. Specifically, the present invention may also be used to locate the position of the animal within the enclosure. For example, the location of each electrode relative to the enclosure may be mapped, using predetermined coordinates, and indexed in a lookup table. Upon the selection by the processor of one or more electrodes for signals to produce the ECG waveform, the coordinates of the animal may be obtained using the lookup table. The coordinates may then be tracked (charted and/or graphed) by software according to the present invention or other software, or output in computer file (e.g., .xls, document format, for example) to be analyzed by other software.

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims.

References

1. Bemtson G G, Bigger J T Jr, Eckberg D L, Grossman P, Kaufmann P G, Malik M, Nagaraja H N, Porges S W, Saul J P, Stone P H, van der Molen M W. Heart rate variability: origins, methods, and interpretive caveats. Psychophysiology. 1997 November; 34(6): 623-48.
2. Bloomfield D M, Zweibel S, Bigger J T Jr, Steinman R C. R-R variability detects increases in vagal modulation with phenylephrine infusion. Am J Physiol. 1998 May; 274(5 Pt 2): H1761-6.
3. Stark R I, Myers M M, Daniel S S, Garland M, Kim Y I. Gestational age related changes in cardiac dynamics of the fetal baboon. Early Hum Dev. 1999 January; 53(3): 219-37.
4. Sahni R, Schulze K F, Kashyap S, Ohira-Kist K, Fifer W P, Myers M M. Maturational changes in heart rate and heart rate variability in low birth weight infants. Dev Psychobiol. 2000 September; 37(2): 73-81.
5. Sahni R, Schulze K F, Kashyap S, Ohira-Kist K, Fifer W P, Myers M M. Postural differences in cardiac dynamics during quiet and active sleep in low birthweight infants. Acta Paediatr. 1999 December; 88(12): 1396-401.
6. Sloan R P, Bagiella E, Shapiro P A, Kuhl J P, Chernikhova D, Berg J, Myers M M.Hostility, gender, and cardiac autonomic control. Psychosom Med. 2001 May-June; 63(3): 434-40.
7. Pine D S, Wasserman G A, Miller L, Coplan J D, Bagiella E, Kovelenku P, Myers M M, Sloan R P. Heart period variability and psychopathology in urban boys at risk for delinquency. Psychophysiology. 1998 September; 35(5): 521-9.
8. Sloan R P, Demeersman R E, Shapiro P A, Bagiella E, Kuhl J P, Zion A S, Paik M, Myers M M. Cardiac autonomic control is inversely related to blood pressure variability responses to psychological challenge. Am J Physiol. 1997 May; 272(5 Pt 2): H2227-32.
9. La Rovere M T, Pinna G D, Hohnloser S H, Marcus F I, Mortara A, Nohara R, Bigger J T Jr, Camn A J, Schwartz P J. Baroreflex sensitivity and heart rate variability in the identification of patients at risk for life-threatening arrhythmias: implications for clinical trials. Circulation. 2001 Apr. 24; 103(16): 2072-7.
10. Bigger J T Jr, Fleiss J L, Steinman R C, Rolnitzky L M, Schneider W J, Stein P K. RR variability in healthy, middle-aged persons compared with patients with chronic coronary heart disease or recent acute myocardial infarction. Circulation. 1995 Apr. 1; 91(7): 1936-43.

1. An apparatus for detecting a signal indicative of at least one of a heart beat, a heart rate, and one or more ECG waveforms of an animal comprising:
a first multiplexer for receiving a signal from each of a plurality of electrodes arranged to permit contact by a part of an animal for a period of time, wherein the first multiplexer includes a first output comprising the signal of a first electrode of the plurality of electrodes;
a second multiplexer for receiving a signal from each of the plurality of electrodes, wherein the second multiplexer includes a second output comprising the signal of a second electrode of the plurality of electrodes; and
a differential circuit for receiving the first output of the first multiplexer and the second output of the second multiplexer, wherein the differential circuit outputs a differential signal, based upon the first output of the first multiplexer and the second output of the second multiplexer, indicative of at least one of a heart beat, heart rate and an ECG waveform of an animal.

2. The apparatus according to claim 1, wherein the first multiplexer and the second multiplexer represent a common multiplexer.

3. The apparatus according to claim 1, further comprising a third multiplexer for receiving the output of the differential circuit and the output of the first multiplexer, wherein the third multiplexer includes a third output comprising either the differential signal or the signal from the first multiplexer.

4. The apparatus according to claim 3, further comprising a processor for controlling the operation of one or more of the first, second and third multiplexers and/or processing the third output signal from the third multiplexer.

5. The apparatus according to claim 4, further comprising an analog to digital converter for converting the output of the third multiplexer to a digital signal for processing and/or analysis by the processor.

6. The apparatus according to claim 4, wherein the processor is capable of analyzing the third output signal to output a signal indicative of at least one of a heart-beat, heart rate and an ECG waveform of an animal.

7. The apparatus according to claim 1, further comprising an amplifier for amplifying a signal from an electrode.

8. The apparatus according to claim 1, further comprising an amplifier for each electrode for amplifying a signal emanating therefrom.

9. The apparatus according to claim 8, wherein the electrodes are positioned adjacent a corresponding amplifier.

10. The apparatus according to claim 1, wherein one or more electrodes are each positioned on a column.

11. The apparatus according to claim 10, wherein one or more of the columns are electrically shielded.

12. The apparatus according to claim 10, wherein the columns are movable.

13. The apparatus according to claim 1, further comprising the plurality of electrodes, wherein the electrodes are positioned within an area and spaced apart from one another a predetermined distance.

14. The apparatus according to claim 13, wherein the predetermined distance comprises a distance to promote the likelihood that a single appendage of the animal will contact a single electrode.

15. The apparatus according to claim 13, wherein the plurality of electrodes form a grid.

16. The apparatus according to claim 1, wherein the differential circuit comprises a differential amplifier.

17. The apparatus according to claim 1, wherein the processor includes an output for sending a signal indicative of at least one of a heart beat, a heart rate and an ECG waveform of an animal in contact with at least two of the electrodes to a computer.

18. The apparatus according to claim 1, wherein the electrodes comprise a silver/silver-chloride alloy.

19. A method for detecting a signal indicative of at least one of a heartbeat, a heart rate, and an ECG waveform of an animal comprising:
scanning each of a plurality of electrodes for a signal indicative of contact by an animal;
selecting a signal from each of at least a pair of electrodes, wherein each selected electrode includes a signal indicative of contact with the animal;
creating a differential signal from the signals of the at least a pair of electrodes; and
determining at least one of a heart beat, a heart rate and an ECG waveform.

20. The method according to claim 19, further comprising extracting ECG waveform parameters from the one or more ECG waveforms.

21. The method according to claim 20, further comprising extracting the variability and/or the coefficient of variability among one or more ECG waveform parameters of a plurality of ECG waveforms.

22. The method according to claim 20, wherein the waveform parameters comprise at least one of P-peak, Q-trough, R-peak, S-trough and T-peak.

23. The method according to claim 20, further comprising extracting interval information between a pair of waveform parameters.

24. The method according to claim 20, wherein the interval information includes at least one interval of the interval between the P and Q parameters, the interval between the P and R parameters, the interval between the P and S parameters, the interval between the P and T parameters, the interval between the Q and R intervals, the interval between the Q and S parameters, the interval between the Q and T parameters, the interval between the R and S parameters, the interval between the R and T parameters, and the interval between the S and T parameters.

25. The method according to claim 19, further comprising providing the plurality of electrodes spaced apart from one another a predetermined distance for contact by an animal.

26. The method according to claim 19, further comprising displaying at least one of the heart beat, the heart rate and the ECG waveform.

27. The method according to claim 26, further comprising displaying at least one of the ECG waveform parameters of the one or more ECG waveforms, intervals therebetween, and the variability and/or the coefficient of variability among one or more ECG waveform parameters of a plurality of ECG waveforms.

28. The method according to claim 19, further comprising amplifying the signal from each electrode.

29. The method according to claim 19, wherein scanning comprising testing each electrode for the presence of a signal indicative of contact on the electrode by a part of an animal.

30. The method according to claim 19, wherein the signal comprises a predetermined level of electrical noise.

31. The method according to claim 19, wherein the signal indicative of contact with an animal comprises an increased level of electrical interference.

32. The method according to claim 19, further comprising filtering at least one of one or more signals from the electrodes and/or the differential signal.

33. The method according to claim 32, wherein filtering comprises filtering out electrical signals of about 50 Hz and/or about 60 Hz.

34. A system for detecting at least one of a heart beat, a heart rate and an ECG waveform of an animal comprising:
scanning means for scanning each of a plurality of electrodes for a signal indicative of contact by an animal;
selecting means for selecting a signal from each of at least a pair of electrodes, wherein each selected electrode includes a signal indicative of contact with the animal;
creating means for creating a differential signal from the signals of the at least a pair of electrodes; and
determining means for determining at least one of a heart beat, a heart rate and an ECG waveform from one or more differential signals.

35. The system according to claim 34, further comprising the plurality of electrodes spaced apart from one another a predetermined distance for contact by an animal.

36. The system according to claim 34, wherein the determining means comprises a processor.

37. The system according to claim 34, wherein any one or more of the scanning means and selecting means comprise a multiplexer controlled by a processor.

38. The system according to claim 34, wherein the creating means comprises a differential circuit.

39. The system according to claim 34, wherein each signal of each electrode is amplified.

40. The system according to claim 38, wherein the differential circuit comprises a differential amplifier.

41. An apparatus for detecting at least one of a heart beat, a heart rate and an ECG waveform of an animals, the apparatus comprising a plurality of electrodes spaced apart from one another a predetermined distance and positioned on columns, wherein each electrode passes a signal indicative of a heartbeat of the animal upon the presence of a part of the animal in contact with an electrode.

42. The apparatus according to claim 41, wherein the plurality of electrodes are retractable.

43. A computer readable medium having computer instructions provided thereon for enabling a computer to perform a method for detecting a signal indicative of at least one of a heart beat, a heart rate, and an ECG waveform of an animal, the method comprising:
scanning each of a plurality of electrodes for a signal indicative of contact by an animal;
selecting a signal from each of at least a pair of electrodes, wherein each selected electrode includes a signal indicative of contact with the animal;
creating a differential signal from the signals of the at least a pair of electrodes; and
determining at least one of a heart beat, a heart rate, and an ECG waveform from the differential signal.

44. An application program operable on a computer system for enabling the computer system to perform a method for detecting a signal indicative of at least one of a heart beat, a heart rate, and an ECG waveform of an animal, the method comprising:
scanning each of a plurality of electrodes for a signal indicative of contact by an animal;
selecting a signal from each of at least a pair of electrodes, wherein each selected electrode includes a signal indicative of contact with the animal;
creating a differential signal from the signals of the at least a pair of electrodes; and
determining at least one of a heart beat, a heart rate, and an ECG waveform from the differential signal.

45. A method for detecting a signal indicative of at least one of a heart beat, a heart rate, and an ECG waveform of an animal comprising:
scanning the plurality of electrodes over a predetermined time period, wherein scanning comprises:
computing the maximum of absolute values of substantially all the electrode signals during the predetermined time period;
determining at least a first pair of electrodes signals having the highest maximum value relative to other electrode signals;
determining whether the signals from the first pair of electrodes greater than a predetermined threshold value; and
determining a differential value from the signals of the first pair of electrodes upon the value of the signals being greater than the threshold;
capturing a plurality of differential values via scanning, wherein the captured differential values represent a waveform; and
processing the waveform.

46. The method according to claim 45, wherein processing comprises:
determining a frequency distribution of the waveform;
comparing the frequency distribution of the waveform to a predetermined frequency distribution of a predetermined ECG waveform; and
comparing the maximum and/or mean amplitude of the waveform to predetermined maximum and/or mean amplitude values of the predetermined ECG waveform upon the frequency distribution of the waveform coming within the frequency distribution of the predetermined ECG waveform;
returning to the capturing step upon the maximum amplitude value corresponding to the maximum amplitude value of the predetermined ECG waveform and/or the mean amplitude value of the waveform corresponding to the mean amplitude value of the predetermined ECG waveform.

47. The method according to claim 45, further comprising returning to the scanning step if the maximum amplitude value fails to correspond to the maximum amplitude value of the predetermined ECG waveform and/or the mean amplitude value of the waveform fails to correspond to the mean amplitude value of the predetermined ECG waveform.

48. The method according to claim 45, further comprising subsequently scanning of the electrodes upon the determination that the signals from the first pair of electrodes are less than a predetermined threshold value.

49. The method according to claim 45, wherein computing of absolute values comprises:
acquiring a sample signal representing a voltage sample from a first electrode;
calculating the absolute value of the sample signal of the first electrode; and
storing the absolute value of the sample signal for the first electrode as a new maximum upon the absolute value of the sample signal being the largest for the first electrode.

50. The method according to claim 45, further comprising displaying the waveform.

51. A computer readable medium having computer instructions provided thereon for enabling a computer system to perform a method for detecting a signal indicative of at least one of a heart beat, a heart rate, and an ECG waveform of an animal, the method comprising:
scanning the plurality of electrodes over a predetermined time period, wherein scanning comprises:
computing the maximum of absolute values of substantially all the electrode signals during the predetermined time period;
determining at least a first pair of electrode signals having the highest maximum value relative to other electrode signals; and
determining whether the signals from the first pair of electrodes are greater than a predetermined threshold value;
determining a differential value from the signals of the first pair of electrodes when the value of the signals is greater than the threshold;
capturing a plurality of differential values via scanning, wherein the captured differential values represent a waveform; and
processing the waveform.

52. An application program operable on a computer system for performing a method for detecting a signal indicative of at least one of a heart beat, a heart rate, and an ECG waveform of an animal, the method comprising:
scanning the plurality of electrodes over a predetermined time period, wherein scanning comprises:
computing the maximum of absolute values of substantially all the electrode signals during the predetermined time period;
determining at least a first pair of electrode signals having the highest maximum value relative to other electrode signals; and
determining whether the signals from the first pair of electrodes are greater than a predetermined threshold value;
determining a differential value from the signals of the first pair of electrodes when the value of the signals is greater than the threshold;
capturing a plurality of differential values via scanning, wherein the captured differential values represent a waveform; and
processing the waveform.
